(19) United States
(12) Patent Application Publication
(10) Pub. No.: US 2013/0115616 A1
(43) Pub. Date: May 9, 2013

(54) DETECTION OF NUCLEIC ACIDS BY AGGLUTINATION

(71) Applicant: Cindy Yamamoto, Irvine, CA (US)

(72) Inventor: Cindy Yamamoto, Irvine, CA (US)

(73) Assignees: HITACHI CHEMICAL RESEARCH CENTER, INC., Irvine, CA (US); HITACHI CHEMICAL CO., LTD., Tokyo (JP)

(21) Appl. No.: 13/809,337

(22) PCT Filed: Jul. 13, 2011
(86) PCT No.: PCT/US2011/043898

§ 371 Date: Jan. 9, 2013

Related U.S. Application Data
(60) Provisional application No. 61/364,758, filed on Jul. 15, 2010.

(51) Int. Cl. C12Q 1/68

(52) U.S. Cl. 435/6.12

(57) ABSTRACT

Embodiments of the invention relate generally to methods detecting, quantifying, or purifying nucleic acids by way of agglutination reactions. Several embodiments amplify target nucleic acids while incorporating a label such as 5-methyl-cytosine into amplified product and detecting, quantifying, or purifying the product with latex beads coupled to antibody reactive to the 5-methyl-cytosine labeled nucleic acid. Several embodiments incorporate DNA labels into specifically designed primers in order to detect, quantify, or purify a product after agglutination.

### RELATED CASES

This application claims the benefit of U.S. Provisional Application Ser. No. 61/364,758, filed on Jul. 15, 2010, the disclosure of which is incorporated in its entirety by reference herein.

### BACKGROUND

1. Field of the Invention

The present disclosure relates to methods of detecting labeled nucleic acids via agglutination reactions. In particular, certain embodiments related to the use of antibodies or oligonucleotides coupled to solid supports, such as latex beads, and to methods of incorporating labels into nucleic acids (e.g., DNA). In several embodiments, the solid support and labeled DNA forms an aggregate that increases turbidity of the reaction mixture and can be detected by various methods, including visual inspection.

2. Description of Related Art

Nucleic acids are basic components of biological systems. Amplification, detection and quantification of nucleic acids are important tools used in research, clinical diagnostics, pharmaceutical, environmental and food industries. A variety of detection methods exist for amplified and non-amplified nucleic acids with some methods capable of quantification.

Currently, real time nucleic acid detection is one of the most sensitive gene analysis techniques available. It is used for a broad range of applications including quantitative gene expression analysis, genotyping, SNP analysis and drug target validation. However, the high cost instrumentation and disposable reagents may limit its use in some industries. Traditional PCR allows amplification of DNA without the higher costs associated with real time detection. Unlike quantitative real time PCR however, results are collected after the reaction is complete. The detection of amplified DNA with traditional PCR is commonly accomplished by agarose gel electrophoresis and use of a nucleic acid intercalating agent, for example, ethidium bromide, which is fluorescent under ultraviolet light. Other dye based systems such as SYBR® Green absorb light then emit a known wavelength of light which can be visualized by gel electrophoresis or. The detection methods are time-consuming, require specialized equipment, and/or present certain health risks. For example, dyes which bind DNA with high affinity raise the concern with respect to carcinogenesis in the user. While newer dyes address some of these concerns, they often require specialized equipment. There is a need for methods of detecting amplified DNA that does not require expensive, complicated equipment, or complicated analysis.

### SUMMARY

In several embodiments, there are provided methods for detecting the presence of an amplified nucleic acid comprising amplifying a sample of a specific target nucleic acid under amplification conditions that incorporate a detectable label into the sample of the target nucleic acid during the amplification, contacting the specific target nucleic acid samples with a sample of a capture matrix, wherein the capture matrix comprises a solid support coupled to an agent that recognizes the detectable label, wherein recognition of the detectable label by the agent induces agglutination of the specific target nucleic acid with the capture matrix and causes an associated increase in turbidity of the specific target nucleic acid sample; and detecting the presence of the amplified target nucleic acid by detecting the increased turbidity in the specific target sample. In several embodiments, the methods further comprise subjecting a sample of control nucleic acid that does not include the specific target nucleic acid to the amplification conditions, contacting the control nucleic acid sample with a sample of a capture matrix, wherein contacting of the control nucleic acid sample with the capture matrix produces a control level of induction of agglutination and associated alteration of turbidity of the sample; and detecting the presence of the amplified target nucleic acid by detecting the increased turbidity in the specific target sample as compared to the control sample. In several embodiments, the methods further comprise quantifying the amount of amplified specific target nucleic acid by comparing the turbidity of the specific target sample with a standard curve. In such embodiments, the standard curve is generated by amplifying a plurality of known amounts of a nucleic acid comprising a detectable label to generate a plurality of samples and contacting each of the plurality of samples with a sample of capture matrix, thereby generating a standard curve of turbidity corresponding to the known amounts of nucleic acid.

In several embodiments, the contacting of the specific target nucleic acid sample with the sample of a capture matrix occurs prior to the amplification. Such embodiments are particularly advantageous because there is no post-amplification manipulation of the samples required to detect the target nucleic acid. For example, the amplification vessel (e.g., a sample tube) need not be opened in order to add the capture matrix. Thus, contamination of the amplified samples is reduced in some embodiments. Also, through-put of the reactions is increased, as the overall number of active steps is reduced. Thus, in certain embodiments, this is advantageous in screening large numbers of samples for certain target nucleic acids.

In several embodiments, the solid support portion of the capture matrix comprises latex beads. In several embodiments, the solid support is coupled to an antibody that interacts with the detectable label. In several embodiments, the detectable label is 5-methyl-cytosine and wherein the antibody is reactive to 5-methyl-cytosine.

In some embodiments, the amplification of the specific target nucleic acid comprises using at least one biotin-labeled primer. In some such embodiments, the method further comprises concentrating the amplified specific target nucleic acid by contacting the amplified specific target nucleic acid with streptavidin coupled to a solid support. In one embodiment, the method further comprises washing the concentrated amplified specific target nucleic acid to remove excess detectable label prior to the contacting with the capture matrix.

In several embodiments, the detectable label are incorporating by amplifying the specific target nucleic acid using one or more modified primers with a tag sequence. In some embodiments, one or more of the modified primers comprises a gene-specific region, a chain terminating nucleotide, and a nucleic acid label comprising 6-20 nucleotides. In some such embodiments, the nucleic acid label is recognized by the agent. In some embodiments, the nucleic acid label comprises a DNA label and the agent comprises DNA at least partially complementary to the DNA label. In several embodiments, the nucleic acid label is selected from the group consisting of oligonucleotide labels, single-stranded DNA labels, RNA labels, peptide nucleic acid labels, locked nucleic acid labels, glycol nucleic acid labels, threose nucleic acid labels, or combinations thereof.

In several embodiments, the nucleic acid is selected from the group consisting of DNA, RNA, siRNA, tRNA, and snRNA.

In several embodiments, the amplification of nucleic acid is performed using nucleotides at a concentration between 1 and 50 μM. In some embodiments, amplification of nucleic acid is performed using nucleotides at a concentration between 1 and 20 μM. In several embodiments, the method of amplification of nucleic acid is selected from the group consisting of the polymerase chain reaction, rolling circle amplification, nucleic acid sequence based amplification, transcription mediated amplification, and ligase chain reaction.

In several embodiments, the detectable label comprises at least one target specific primer comprising at least one 2′ O-methyl RNA base, and the agent comprises a single stranded nucleic acid complementary to a region on the target specific primer. In some such embodiments, the single stranded nucleic acid is selected from a group consisting of oligonucleotides, single-stranded DNA, RNA, peptide nucleic acid, locked nucleic acid, glycol nucleic acid, and threose nucleic acid. In one embodiment, the solid support is latex beads and at least one target specific primer is coupled to the solid support.

### BRIEF DESCRIPTION OF THE FIGURES

FIG. 1. Detection of 5-methyl-cytosine. Step (A) depicts the amplification of DNA with a methylated cytosine (small filled circle represents 5-methyl-cytosine) by either PCR (left) or an isothermal amplification method (right). Step (B) depicts the addition of anti-5-methyl-cytosine labeled latex beads.

FIG. 2. Detection of 5-methyl-cytosine in PCR using magnetic beads. Step (A) depicts PCR amplification of DNA that includes a labeled cytosine nucleotide and employs a single biotin-labeled primer. Step (B) involves the addition of streptavidin-magnetic beads and removal (by washing) of excess 5-methyl-cytosine. Step (C) is the addition of addition of anti-5-methyl-cytosine labeled latex beads. Step (D) depicts the use of a magnet to visualize the agglutination of DNA that has 5-methyl-cytosine incorporated and reacted with the streptavidin-magnetic beads.

FIG. 3. Detection of DNA using modified primers with ‘DNA label’. Step (A) depicts PCR amplification of DNA using modified primers comprising a chain termination nucleotide analog and a DNA label (comprising 6-20 nucleotides). Step (B) depicts the addition of oligo-modified latex beads, which allow agglutination to occur.

FIGS. 4A-4I depict the results of an agglutination assay of PCR samples using detection of 5-methyl-cytosine based on the amount of starting template for the PCR reaction. 4A) 13 ng; 4B) 1 ng; 4C) 0.1 ng; 4D) 0.01 ng; 4E) 0.001 ng; 4F) 0.1 μg; 4G) 0.01 pg; 4H) no template (e.g., control reaction); 4I) no methyl cytosine (e.g., control reaction).

FIGS. 5A-5H depict the result of an agglutination assay using modified primer PCR samples and detection through the use of a ‘DNA label’. Results are based on the number of copies of template DNA. 5A) 10⁸ copies; 5B) 10⁷ copies; 5C) 10⁶ copies; 5D) 10⁵ copies; 5E) 10⁴ copies; 5F) 10³ copies; 5G) 10² copies; 5H) no template (e.g., control reaction).

FIGS. 6A-6C depict the results of an agglutination reaction performed on DNA with and without single-stranded ends, or with double stranded ends. FIG. 6A shows the results of agglutination of DNA having single stranded ends. FIG. 6B shows the results of agglutination when no DNA label is incorporated during amplification. FIG. 6C shows the results of agglutination of DNA having double stranded ends.

FIGS. 7A-7C depict the results of a agglutination assays using padlock probes (with various Let 7D (a tumor suppressor microRNA) sequences) and rolling circle amplification with hybridization to oligo labeled latex beads. FIG. 7A shows the results when the Let 7D primers are used, and agglutination is observed. FIGS. 7B and 7C show the agglutination results when a Let 7D mutant primer or a Let 7d-ligase primer is used and little to no agglutination is observed.

### DETAILED DESCRIPTION

There is a need for methods of detecting amplified DNA that does not require expensive, complicated equipment, or complicated analysis. Microsphere agglutination, as disclosed herein, is a visual and low complexity nucleic acid detection method for molecular diagnostics. Several embodiments of this invention provide an immediate visual confirmation of the presence of a target nucleic acid (e.g., DNA) after an amplification step (e.g., PCR). The methodology can be applied to detect the presence of target nucleic acid that is present in an industrial or clinical sample such as drinking or ground water, food, blood etc. Embodiments of the invention provide an inexpensive, fast, and simple method for detecting amplified nucleic acids without requiring the use of gel electrophoresis, fluorescent dyes such as ethidium bromide, or specialized detection equipment. Dyes such as ethidium bromide potentially expose the operator to the mutagenic effects of the dye as well as exposure to ultraviolet light. Several embodiments are compatible with detection of increased sample turbidity during or after amplification by analysis with diffuse transmission optics. An advantage of these methods is that the amplification reaction may also be visualized by the naked eye and compared to a series of standards or controls that display a range of turbidity. In some embodiments, a control is a solution subject to the same amplification conditions as the experimental sample, but known to contain no amplified product. After mixing with a capture matrix according to embodiments disclosed herein, such a control can be used as the basis of identifying the presence or absence of an amplified target product. In some embodiments, known amounts of nucleic acid are amplified and combined with the capture matrix, allowing the generation of a standard curve. Thus, in some embodiments, agglutination controls are used to assess the level of nucleic acid amplification. The methods disclosed herein thus provide simple and inexpensive determination of the presence of a target sequence, or quantification of the target without further manipulation of the sample. In several embodiments, the methods further allow the isolation of the target product.

Electrophoresis of nucleic acids can be cumbersome and time-consuming because multiple sample manipulations may be required. For example, in order to analyze nucleic acids, desiccated agarose is heated in a suitable buffer then allowed to cool and set. A dye, such as ethidium bromide, is added to the agarose mixture, the DNA sample or the buffer. Each DNA sample of interest is mixed with a viscous liquid to facilitate loading into the wells of the agarose gel. The sample loading solution may also contain dyes to indicate the progress of the sample DNA through the gel so that the operator can terminate electrophoresis when the DNA has travelled a suitable distance. The DNA sample is then visualized under UV light and the gel may be photographed with a digital camera. In several embodiments described herein, this extensive protocol is avoided by virtue of visual inspection of a PCR tube being indicative of the presence of target PCR product.

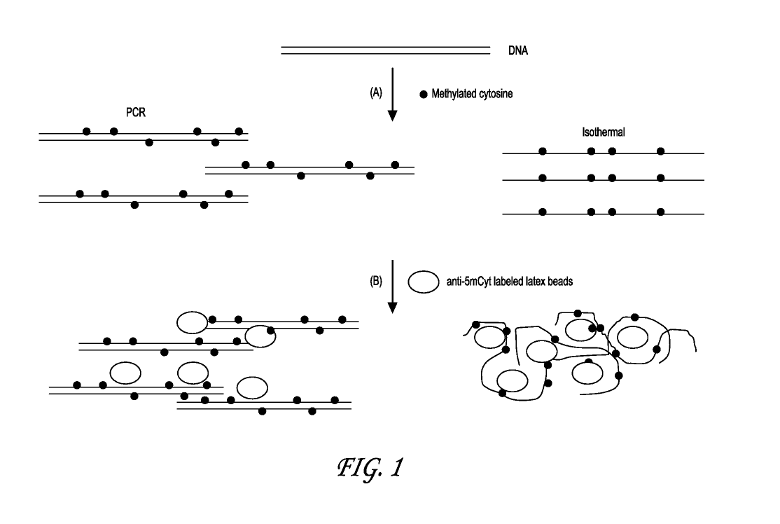

In some embodiments, the amplification reaction incorporates a label into the amplified product which is then detected by the binding of a label-specific antibody or antibody-like molecule bound to a solid support or matrix (e.g., a capture matrix). As used herein, the term “solid support” shall be given its ordinary meaning and shall also refer to antibody-labeled or oligo-labeled latex beads or other immobilized or recoverable antibody or oligo-labeled surface (e.g., a labeled filter or a sample tube/well comprising a label). The interaction between the amplified target and antibody results in aggregation of the matrix which can be observed as increased turbidity by the naked eye. The label may be incorporated into any nucleic acid such as, for example, double or single stranded RNA or DNA. Any suitable amplification method may be used such as, for example, PCR, RCA, LAMP, NASBA, TMA etc. In some embodiments the amplified nucleic acid is detected by incorporation of a labeled (or otherwise detectable) nucleotide into the nucleic acid. For example, in some embodiments, 5-methyl-cytosine is incorporated into DNA, such as into PCR product, followed by aggregation or agglutination with a solid support structure such as latex beads coupled to antibody that is reactive to the 5-methyl-cytosine. The increase in turbidity from the resulting aggregation of DNA and latex, which can be detected by the naked eye, spectrophotometry, turbidometry or nephelometry, is indicative of both the presence of the target DNA of interest and of a successful amplification reaction (FIG. 1, left).

Incorporation of 5-methyl-cytosine may be achieved in a PCR reaction by using the same reaction mixture as used with standard PCR with the exception of a methylated cytosine. The addition of 5-methyl-cytosine can completely replace non-labeled CTP in these reactions. In contrast to a detection system that uses labeled primer pairs, incorporation of the methyl label, in potentially all cytosine positions, yields a higher density of label in the target PCR product. Thus, some embodiments of this method are advantageously more sensitive. This increased sensitivity can be used to detect PCR product at an earlier stage of cycling and detection can be achieved without using multiple steps (as is required with many existing nucleic acid detection techniques). Thus, several embodiments of the invention are more efficient with respect to time for analysis. The levels of non-labeled and labeled nucleotides can be reduced if detection of DNA directly following amplification reaction is desired. Low levels of non-labeled and labeled dNTPs are typically beneficial in reducing binding of antibody-coated particles to excess labeled dNTP in the final reaction mixture. In several embodiments, the concentration of dNTPs used in amplification reactions ranges from about 1 to about 50 μM. In several embodiments, the concentration of dNTPs ranges between about 1 to about 10 μM, about 10 to about 20 μM, about 20 to about 30 μM, about 30 to about 40 μM, or about 40 to about 50 μM, and overlapping ranges thereof. In several embodiments, the concentration of dNTPs used is greater than 50 μM. In some embodiments, about 1 to about 10 μM, including 2, 3, 4, 5, 6, 7, 8, and 9 μM results in substantial reduction of binding of antibody-coated particles to excess labeled dNTP. Longer extension times during the amplification may increase efficiency of the amplification reaction and sensitivity of the assay.

In some embodiments, nucleic acid sequence base amplification (NASBA) is used to amplify RNA (FIG. 1, right). In brief, RNA is hybridized to a complementary first primer at the 3′ end of the RNA. Then, reverse transcriptase synthesizes a complementary DNA (cDNA). Added RNAase H degrades RNA when it is in a DNA-RNA hybrid, thus RNAse H degrades the RNA strand leaving only the cDNA. A second primer is thereafter hybridized to the cDNA strand. The second primer acts as a primer for T7 RNA polymerase which produces multiple copies of RNA which can be used as templates for the first primer and so on. The rounds of NASBA amplification are based on the hybridization of a primer to an RNA template which is ultimately degraded by RNAse H and also by the second primer being incorporated into cDNA which is then used as a template to make more RNA. During the NASBA amplification there is no step requiring high temperature denaturation of double stranded DNA. As such, NASBA is compatible with using a single incubation temperature and thus does not require temperature cycling equipment. In several embodiments, a labeled nucleotide is incorporated into the amplified nucleic acid. In some embodiments, the label comprises a methyl group. In some embodiments the methyl group is linked to a cytosine, (e.g., 5-methyl-cytosine). In some embodiments, the label is incorporated into DNA, while in other embodiments it is incorporated into RNA, and in still other embodiments, it is incorporated into both DNA and RNA.

In some embodiments, transcription mediated amplification (TMA) is used to amplify the target RNA and incorporate 5-methylcytosine. Briefly, a first primer encoding a promoter sequence for RNA polymerase is hybridized to the target RNA at a specific target site. Reverse transcriptase is then used to create a cDNA copy by extension of the promoter primer. The RNA in the resulting RNA:DNA duplex is degraded by RNase. A second primer is hybridized to the cDNA creating a double-stranded DNA molecule which is recognized as a promoter by RNA polymerase. The RNA polymerase initiates transcription to generate multiple copies of the original RNA. Each new RNA molecule then becomes a template for a new round of amplification. In several, embodiments, the TMA reaction is performed isothermally.

In some embodiments, rolling circle amplification (RCA) is used to amplify the target RNA and incorporate 5-methylcytosine. For example, a single stranded DNA probe with 5′ and 3′ ends complementary to a specific region of the target can be circularized with T4 DNA ligase (or T4 RNA ligase II, or another suitable enzyme) when hybridized to the target. This resulting circular probe can be used as the template for RCA. The nucleic acid target can be used as the primer to initiate RCA on the circular probe. RCA will produce 5-methylcytosine incorporated long, single stranded products that can bind to antibody-coated latex particles.

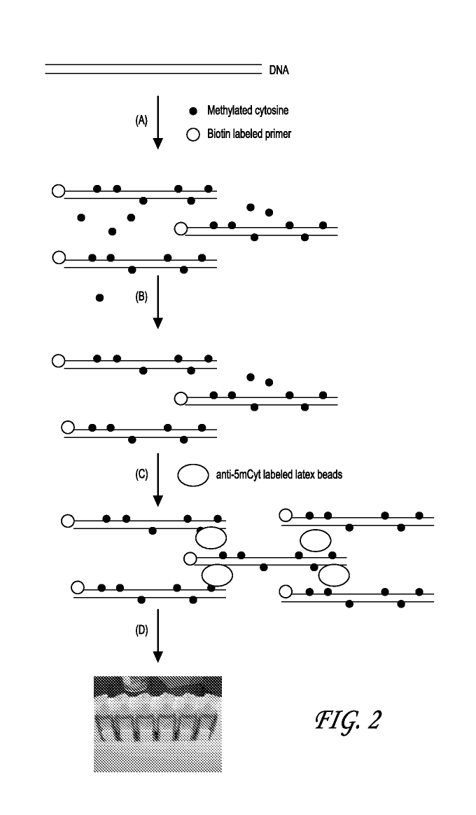

In several embodiments, the nucleic acid target is amplified using at least one labeled nucleotide and at least one labeled primer such as a biotinylated primer (FIG. 2). Following amplification, in some embodiments, biotin-labeled product is localized or concentrated by a streptavidin-coated magnetic bead. Such an approach is advantageous because excess labeled nucleotide is removed through washing the biotin-labeled product-streptavidin-coated bead complex, which improves reaction sensitivity. In several embodiments, a small amount of buffer is used to resuspend the washed amplified product which would be used for the agglutination reactions. In some embodiments, antibody-coated colored particles are added to the washed sample. Any agglutination that occurs is then localized to a concentrated area when the sample is placed in a magnet. A clearing of the sample, due to the magnetic bead-agglutinated product complex will only be visualized if target has been amplified. Thus, the clearing of a turbid sample can be used to determine the presence or absence of a target nucleic acid.

In some embodiments, the nucleic acid target is amplified using modified hairpin primers. Primers potentially form secondary structures such as folding over to form double stranded regions. These internal folds, or “hairpins,” result from base-pairing between nucleotides within the single stranded DNA. In some cases hairpins in primers reduce the efficiency of an amplification reaction, while in some cases, they can be employed to increase the specificity of primer binding. However, in several embodiments, the hairpins were designed to prevent the ‘tag’ sequence from the excess free primers from being exposed and binding to the oligo-labeled particle following the amplification reaction. This approach allows detection of the target directly following amplification and precludes an additional purification step. In some embodiments, the hairpin primers may be composed of a gene-specific region, a 2′ O-methyl RNA base, a 15-20 base pair tag sequence and a 5-10 base pair long sequence to complete the stem.

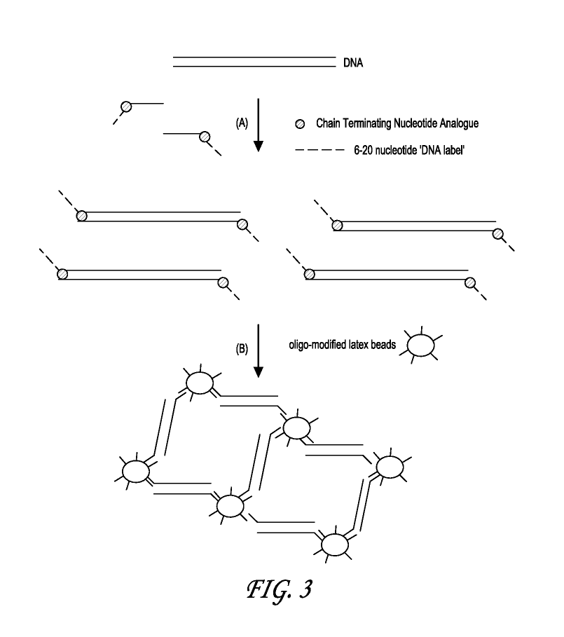

In some embodiments, the nucleic acid target is amplified using modified primers with a tag sequence which will be called ‘DNA label’ (FIG. 3). While the term ‘DNA label’ is used in certain embodiments, in other embodiments, a ‘nucleic acid label’ is used. As used herein, the term ‘nucleic acid label’ shall be given its ordinary meaning and shall also refer to any of oligonucleotide labels, single-stranded DNA labels, RNA labels, peptide nucleic acid labels, locked nucleic acid labels, glycol nucleic acid labels, threose nucleic acid labels, or combinations thereof. In some embodiments, the modified primers may be composed of a gene-specific region, a chain terminating nucleotide analogue, and a 6-20 base pair DNA label.

Amplification using the modified primers produces amplicons with single stranded regions or “handles” at the 5′ and 3′ ends. These single stranded regions are created during amplification with thermophilic DNA polymerase because the polymerase is unable to read through the chain terminating nucleotide analogue. Chain terminating nucleotide or nucleoside analogues can include but are not limited to 2′-O-methyl nucleosides, 2′-C-methyl nucleosides, β-D-N⁴-hydroxycytidine, N⁴-amino-5,6-dihydrocytosine-6-sulfonate, N⁴-methoxy-5,6-dihydrocytosine-6-sulfonate, 2-chloro-2′-deoxyadenosine, 3′-deoxyribonucleosides, azidothymidine and 2′,3′-dideoxynucleosides. Thus, the DNA label will be available for hybridization to DNA having complementary sequences to the label. In several embodiments, a solid support (e.g., latex beads) is provided that is coupled to this complementary DNA which will hybridize to the amplified nucleic acid causing agglutination. In some embodiments, the solid support is coupled to single stranded oligonucleotides.

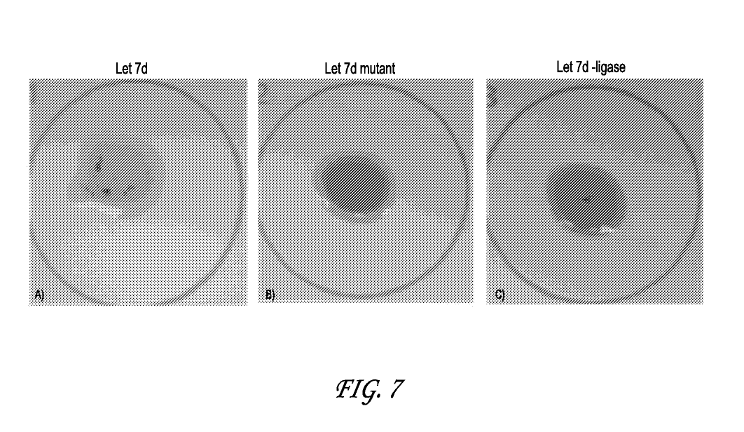

In some embodiments, the nucleic acid target is amplified by a method such as RCA and is detected by latex beads coated with DNA having complementary sequences to regions of the single stranded product. For example, a single stranded DNA probe with 5′ and 3′ ends complementary to a specific region of the target can be circularized with T4 DNA ligase (or T4 RNA ligase II, or another suitable enzyme) when hybridized to the target. This resulting circular probe can be used as the template for RCA. The nucleic acid target can be used as the primer to initiate RCA on the circular probe. RCA will produce long, single stranded products that can hybridize to sequences on the DNA-coated particles (FIG. 7).

An unexpected advantage of certain embodiments is that the capture matrix (e.g., latex capture beads) can be added to the PCR reaction so that amplification of the target DNA and detection of the PCR product can occur simultaneously. This is possible because, unlike protein or enzyme based detection systems, DNA is not destroyed by the high temperatures used during PCR cycling. The target DNA and the capture matrix are merely separated at the peak PCR temperature with subsequent re-hybridization between the target and latex occurring at the annealing temperature. Despite the action of the latex binding to the target at each cycle of the PCR reaction, the amplification reaction kinetics are not substantially changed, and do not interfere with efficient amplification of low copy number target sequences.

Previous attempts to detect PCR products have added a capture reagent after the PCR reaction is complete. These techniques typically require additional sample manipulation to make the double stranded PCR product accessible for hybridization. For example, the PCR product is mixed with a denaturing solution such as NaOH to separate the DNA strands and make them accessible to a capture reagent that contains DNA which is complementary to the target. Then the capture reagent is added under conditions that promote hybridization and capture, typically by diluting and neutralizing the NaOH. The resulting captured DNA may be assessed visually or colorimetrically. In contrast to these methods, several embodiments of the current invention have the advantage of requiring fewer steps and simplifying target detection. This can be achieved, for example, by adding the solid support during the reaction. These embodiments also provide the advantage that reactions involving samples with high concentrations of target can be terminated earlier, at low cycle number, because they can be visually inspected while the reaction is taking place. In many embodiments, this represents a significant saving of time.

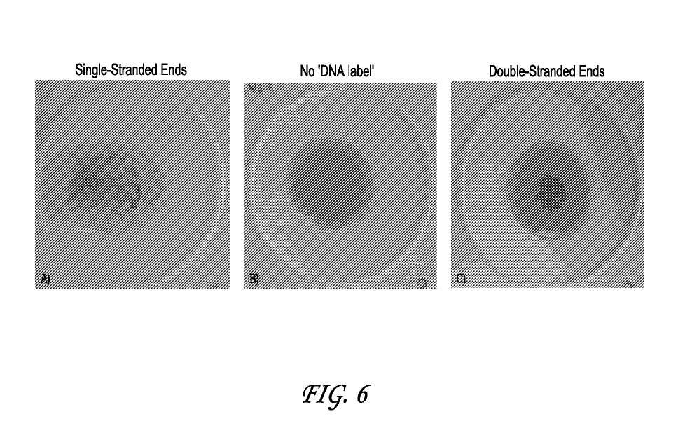

In other embodiments of the invention, solid support coupled complementary DNA are added after the PCR reaction. Unlike PCR reactions with conventional forward and reverse primers, some embodiments of this invention employ modified primers which yield PCR product that has single stranded “handles” at the two termini of the product (FIG. 6). As such, the handles are immediately available for hybridization to the latex beads without a denaturation step. In several embodiments, detection is simplified because there is no need to add denaturants which will subsequently require neutralization. For example, latex beads may simply be added to the PCR tube which is incubated at room temperature. Thus, multiple steps are eliminated saving not only operator time, but also the expense of additional chemical reagents.

Another embodiment of the invention, is directed toward the creation of a visible line of detection on a lateral flow assay. In one such embodiment, the biotin and 5-methyl-cytosine labeled amplicon is added to anti-5-methyl-cytosine coated microparticles, then applied to the lateral flow device. The amplicon bound to the anti-5-methyl-cytosine coated microparticles wicks through the membrane to the streptavidin line on the lateral flow device and is captured by the interaction of biotin and streptavidin. The result is a visible line of agglutinated microparticles.

In some embodiments the solid support may include colored/non-colored latex or other non-latex solid material such as plastic, carbon, polyacrylamide, metals, magnetic material, nitrocellulose, nylon, and/or glass. Particles are preferably of size and shape that allow them to remain in suspension in a solution in the absence of hybridization complex formation. For example, the particles generally have a maximum dimension of between about 0.1 μm and about 1.0 μm. In some embodiments, the particles range between about 0.1 and 0.2 μm, between about 0.2 to 0.4 μm, between about 0.4 and 0.6 μm, between about 0.6 and 0.8 μm, between about 0.8 and 1.0 μm and overlapping ranges thereof. In some embodiments, the particles are preferably between about 0.3 μm and 0.6 μm. The particles can be attached using standard techniques to a probe or antibody by covalent linkage, adsorption, chelation, ionic interactions, or through use of a binding partner set (i.e., ligand and hapten).

Some embodiments of this invention are used to detect nucleic acid methylation. In some embodiments, DNA is treated with bisulfate which coverts cytosine residues to uracil, but does not convert cytosine residues that are methylated at the carbon-5 position. In such embodiments, the methodology can be applied to bisulphite-conversion DNA methylation analysis or “bisulphite sequencing.” One form of bisulphite sequencing involves methylation-specific PCR (MSP). Briefly, MSP involves use of primers designed to be complementary to a region of DNA that has a cytosine base. The primer will have a 3′ terminus that is complementary to the target cytosine. The primer can be modified to incorporate the chain terminating nucleotide analogue and DNA label. If the cytosine has been converted to uracil, a primer designed to bind at that position will not bind and there will be no PCR reaction. Alternatively, if the cytosine is methylated, and thus unchanged by the bisulphite treatment, the primer will anneal and a PCR product will be generated. A PCR reaction utilizing a tag, such as single stranded DNA label or 5-methylcytosine, results in a PCR product that has a ‘handle’ or (methyl) tag incorporated into the DNA. Such a tag can be recognized by a solid support coupled to, for instance, oligonucleotide sequence complementary to the ‘handle’ or 5-methyl-cytosine-specific antibody. Therefore, several embodiments of the invention provide a simple method of generating information about DNA methylation at specific locations without the need for gel electrophoresis or DNA sequencing the PCR products, as presence of the target PCR product can be detected visually.

In some embodiments, regions of naturally methylated DNA are captured with latex beads that are coupled to antibody that is reactive to 5-methyl-cytosine. Briefly, genomic DNA, or RNA, is purified using any suitable technique. Genomic DNA is then fragmented using, for example, sonication or restriction enzyme digestion. Solid support coupled to anti-5-methyl-cytosine antibody are incubated with the fragmented DNA, or RNA. After washing the solid support to remove non-specifically bound nucleic acid and reactants, the remaining nucleic acid is harvested as a fraction that is enriched for methylated DNA and/or RNA.

In some embodiments, methylated DNA is bound to the solid support as outlined above. Then, various cell lysates or cellular fractions are incubated with thesolid support. During the incubation period, proteins that interact with the bound fraction of DNA are also closely associated with the solid support. The resulting aggregation is washed to remove non-specifically bound materials. The DNA associated proteins can be retrieved by dissociating the DNA-protein bond and the enriched protein can be fractionated and/or sequenced. This solid support-DNA matrix can also be applied to any non-protein interaction to enrich samples for DNA associated carbohydrates, lipids, peptides etc. This method is also applicable to RNA.

In some embodiments, controls are used which may consist of amplification reactions of house keeping or constitutively expressed genes such as β2-microglobulin, glyceraldehydes 3-phosphate dehydrogenase (GAPDH), or β-actin. In other embodiments controls consist of known amounts of added synthetic target nucleic acid. In still other embodiments, controls consist of ribosomal RNA, transfer RNA, snRNA, microRNA, and other non-coding RNAs. In other embodiments, controls consist of genomic DNA of known copy number. In some embodiments, controls consist of mRNA that is expressed at a known level or amount.

In some embodiments, controls consist of a solid support (e.g. latex) bound to an antibody that is reactive to 5-methyl-cytosine cytosine or nucleotide sequence that is complementary to the DNA label. The solid support may be mixed with amplification reagents or a completed amplification reaction mixture that lacks a target DNA and/or RNA sequence. In some embodiments, controls consist of amplification reactions that have varying known amounts of target DNA and/or target RNA mixed with the solid support. In other embodiments, controls consist of cards that present images of a lack of agglutination or aggregation. In some embodiments, controls consist of cards representing varying concentrations of target nucleic acid and varying degrees of agglutination or aggregation of the solid support. In other embodiments, controls represent predetermined values of nephelometer or turbidometer readings.

Several embodiments are used to quantify nucleic acids. In some embodiments, a standard curve is generated by combining known amounts of labeled nucleic acid with the capture matrix as described herein. After an amplification reaction is performed, the turbidity or agglutination in the experimental sample is compared to the standard curve to quantify the amount of target nucleic acid that was amplified. In some embodiments, the quantification is made by visual comparison of the experimental sample to the standard curve. In some embodiments, a spectrophotometer or turbidometer is used to measure the absorbance or turbidity of the experimental sample, thereby allowing comparison with the standard curve. In some embodiments, the amount of target nucleic acid is determined by detection of agglutination at a particular number of amplification cycles. For example, the appearance of turbidity after twenty rounds of amplification in a first sample compared to appearance of turbidity after thirty rounds of amplification in a second sample indicates a greater amount of target in the first sample. Thus, quantification can be achieved by comparing experimental samples with control samples of known amounts of target that have been subjected to various rounds of amplification.

In several embodiments, the methods described herein are used to purify a target nucleic acid. In some embodiments, after an amplification reaction has been performed, the resultant amplification solution (e.g., a solution after PCR is complete) is combined with the capture matrix as described above. After an incubation time sufficient to allow interaction of the label incorporated into the target nucleic acid with the capture matrix, the nucleic acid-matrix product can be separated from the remainder of the solution (which may include undesirable nucleic acids, reaction reagents that may inhibit further processing, etc.). In some embodiments, the capture matrix is in solution (e.g., antibody labeled beads). In such embodiments, centrifugation, filtration, sedimentation (or other like methodologies) are used to separate the nucleic acid-matrix product from the remainder of the solution. For example, after a PCR reaction, the PCR product (which comprises the labeled target nucleic acid) is mixed with antibody-labeled or oligo-labeled latex beads in an incubation vessel. After an incubation period, the mixture is centrifuged, thereby causing the target nucleic acid bound to the beads to pellet to the bottom of the incubation vessel. Thereafter the supernatant (with undesired products and/or reagents) is decanted. The nucleic acid-bead product is then washed and subjected to further processing (depending on the experimental use envisioned).

In some embodiments, the capture matrix is coupled to or comprises a solid support. In such embodiments, an amplification product is passed over the matrix, allowing the labeled target nucleic acid to interact with the matrix. Undesired products or excess reaction reagents will pass (or flow through or past) the capture matrix, and are thereby removed. For example, after a PCR reaction, the PCR product (which comprises the labeled target nucleic acid) is loaded onto a column comprising antibody-labeled or oligo-labeled latex beads. As the product passes through the column, the target nucleic acid binds to the capture matrix while the remaining constituents of the solution pass out of the column. The column containing the nucleic acid-capture matrix product is then washed and subjected to further processing (e.g., elution, etc. depending on the experimental use envisioned).

As used herein, the terms aggregation or agglutination shall be given their ordinary meaning and shall also refer to the aggregation, turbidity, structure, complex, or bond formed between the nucleic acid and a solid support structure, such as latex beads. Agglutination may be mediated by protein:nucleic acid, antibody:nucleic acid, DNA:RNA, RNA:RNA, or DNA:DNA hybridization. In some embodiments aggregation or agglutination may involve antibody:antigen binding or antibody:ligand binding. The antibody component may be, but is not limited to, antibody fragments, Fab, F(ab′)2, single-chain variable fragment, chemically linked Fab, bi-specific T-cell engager, synthetic antibodies, aptamers, plastic antibodies or MHC tetramers.

### EXAMPLES

Specific embodiments will be described with reference to the following examples which should be regarded in an illustrative rather than a restrictive sense.

### Example 1

### Detection of PCR Product with 5-methylcytosine

The method as applied to PCR provides a simple, inexpensive, and rapid method of detecting and quantifying PCR products. A variety of A variety of thermophilic DNA polymerases are compatible with the incorporation of 5-methyl-cytosine into DNA. The method can be applied to allele-specific PCR, assembly PCR, asymmetric PCR, hot-start PCR, intersequence-specific PCR, ligation-mediated PCR, Inverse PCR, multiplex-PCR, nested PCR, quantitative PCR etc. The method is also compatible with amplification of RNA.

In one embodiment, a template is incubated in 20 mM Tris-HCl (pH 8.4), 50 mM KCl, 1.5 mM MgCl2, 1 μM dATP, dTTP, dGTP+methyl-dCTP, 0.25 μM each forward and reverse primer, 2.5 Units Taq Polymerase. The sequences of the primers used are as follows:

a) Forward primer -
(SEQ ID No. 1)
5′CGTCCATCTATTTGCCAGGT3′
and
b) Reverse primer -
(SEQ ID No. 2)
5′ATTTCGGATAAAGCGTGGTG3′.

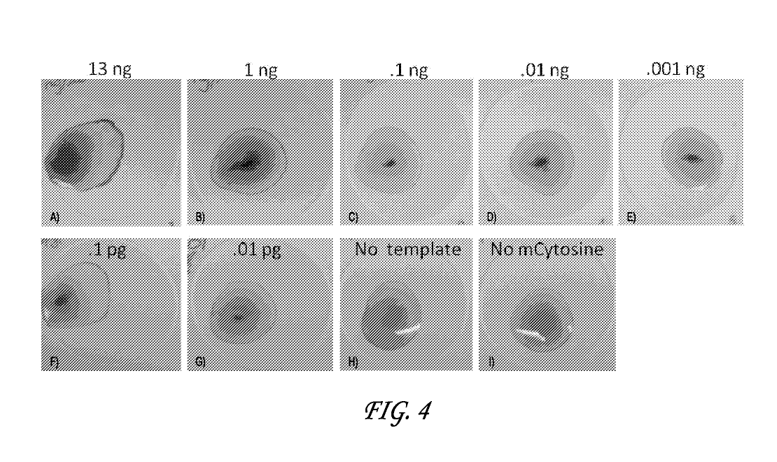

The target nucleic acid is a plasmid containing the cDNA of the Listeria monocytogenes hemolysin gene (Genbank accession no. FJ263386). In several embodiments, a PCR reaction proceeds for 25-35 cycles of 94° C. for 45 seconds, 55° C. for 30 seconds, 72° C. for 2-4 minutes. Other reaction cycles are used in other embodiments, depending on primer design and target characteristics. An aliquot of the reaction (50 uL) is mixed with 6.25 mg/mL latex-antibody conjugate (5 uL) for 5 minutes at room temperature. Alternatively, the latex beads are added to the PCR tube. The resulting aggregation, which is indicative of the presence of PCR product, is inspected visually on cards (see e.g., FIG. 4) or measured in a turbidometer or nephelometer to detect the presence of a target nucleic acid. In other embodiments, reagent concentrations, incubation times, and temperatures vary according to the optimal conditions required for the particular primer sequences, target DNA, and/or RNA. For example, one example of an alternative embodiment is a PCR reaction for 30 cycles of 94° C. for 60 seconds and 61° C. for 2.5 minutes.

### Example 2

### Detection of PCR Product with 5-methyl-cytosine using Biotinylated Primers and Magnetic Beads

Biotinylated primers can be incorporated during the amplification to facilitate the removal of excess 5-methyl-cytosine following completion of the reaction. In this case, a template is incubated in 20 mM Tris-HCl (pH 8.4), 50 mM KCl, 1.5 mM MgCl2, 1 μM dATP, dTTP, dGTP+methyl-dCTP, 0.25 μM biotinylated forward primer, 0.25 μM reverse primer, 2.5 Units Taq Polymerase. A PCR reaction proceeds for 25-35 cycles of 94° C. for 45 seconds, 55° C. for 30 seconds, 72° C. for 2-4 minutes. Other reaction cycles are used in other embodiments, depending on primer design and target characteristics. The reaction is mixed with 0.3 μm streptavidin-coated magnetic beads for 5 minutes, then placed in a magnetic stand for 1 minute. Supernatant, excess biotinylated primer and 5-methyl-cytosine are removed and washed with phosphate buffer. The pellet is resuspended in 20 μL phosphate buffer and mixed with colored latex-antibody conjugate for 5 minutes at room temperature in a microfuge tube. The mixture is then placed in a magnetic stand for 1 minute. The agglutinated products will be localized to the area of the magnet based on the interaction of the biotin on the PCR products and the streptavidin coated on the magnetic beads. The presence or absence of target nucleic acid can be determined based on the positive or negative clearing observed.

### Example 3

### Detection of NASBA Product with 5-methyl-cytosine

The methods described herein can also be applied to RNA based amplification techniques, such as nucleic acid sequence-based amplification.

Briefly, RNA is added to NASBA buffer and pre-incubated at 65° C. for 5 min before incubation at 41° C. for 5 min. An enzyme mix is then added and the reaction is incubated at 41° C. for 90 min. The final concentrations in the NASBA buffer are 40 mM Tris HCl pH 8.5, 12 mM MgCl₂, 70 mM KCl, 5 mM dithiothreitol, 15% dimethyl sulphoxide, 1 mM of each dNTP+methyl-dCTP, 2 mM of ATP, CTP and UTP, 1.5 mM GTP, 0.5 mM ITP and 10 pM of each primer. Primer sets are added in accordance with the desired target nucleic acid. In other embodiments, greater or lesser concentrations of reagents and greater or lesser temperatures and reagent concentrations are employed. The NASBA product is mixed with the latex-antibody conjugate as discussed above. An increase in turbidity is indicative of a positive reaction.

### Example 4

### Detection of PCR Product Using Modified Primers

Some embodiments of the method use latex beads coupled to single stranded DNA which is complementary to a region designed as part of the 5′ end of a gene-specific primer. The complementary region, which is coupled to the latex bead, captures the PCR product which incorporates the corresponding sequence provided by the primer. In some embodiments, the target nucleic acid is incubated in 10 mM Tris-HCl (pH 8.8), 50 mM KCl, 0.8% Nonidet P40, 1.25 mM MgCl2, 0.2 mM dNTP, 0.5 μM each forward and reverse 2′ O-methyl RNA base primers, 2.5 Units Taq Polymerase. The sequences of the primers used are as follows:

a) Lis221F -
(SEQ ID No. 3)
5′-AAAAAAmATCACTCTGGAGGATACGTTGC-3′
and
b) Lis221R -
(SEQ ID No. 4)
5′-AAAAAAmATTACCGTTCTCCACCATTCC-3′.

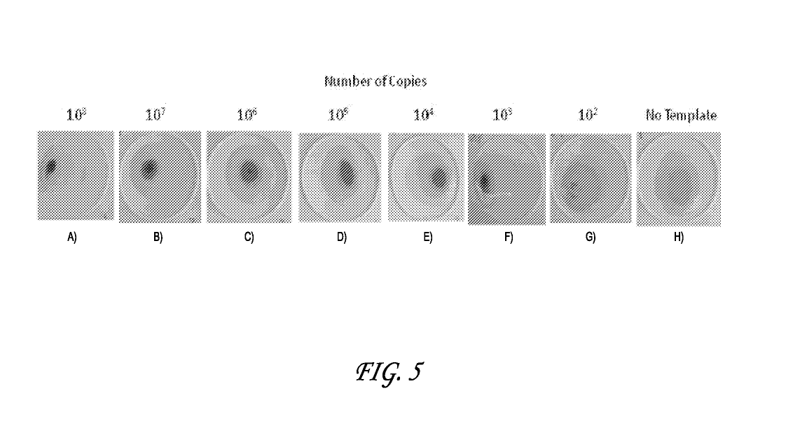

The sequence of the complementary capture probe on the latex bead is 5′-TTTTTTTTTTTTTTTTTTTT-biotin3′ (SEQ ID No. 5). The target nucleic acid is a plasmid containing the cDNA of the Listeria monocytogenes hemolysin gene. (Genbank accession no. FJ263386). In several embodiments, PCR reaction are performed using 25-35 cycles of 94° C. for 45 seconds, 55° C. for 30 seconds, 72° C. for 5 minutes. As discussed above, other PCR reactions are used in some embodiments, depending on the primers used and the desired target sequence. An aliquot of the reaction can then be mixed with the latex-complementary nucleic acid sequence reagent for, in some embodiments, 5 minutes at room temperature. In other embodiments, reagent concentrations, incubation times, and temperatures will vary according to the optimal conditions required for the particular primer sequences, target DNA, and/or RNA. For example, a PCR reaction may proceed for 45 cycles of 96° C. for 60 seconds, 60° C. for 45 seconds, 72° C. for 1 minute. For the latex agglutination assays, 30 uL of the PCR sample and 5 uL of the 6.25 mg/mL oligo-latex bead was applied to the latex card and mixed with a pipet tip. The resulting aggregation, which is indicative of the presence of PCR product, is inspected visually, on a card (see FIG. 5), or measured in a turbidometer or nephelometer to detect the presence of a target nucleic acid.

### Example 5

### Latex Agglutination Reaction with and without Single-Stranded Ends

Agglutination reactions may also be optimized based on the methods disclosed herein. For example, the rate of agglutination was significantly improved when single stranded ends were available for hybridization on PCR products. In this example, the target nucleic acid was incubated in 10 mM Tris-HCl (pH 8.8), 50 mM KCl, 0.8% Nonidet P40, 1.25 mM MgCl2, 0.2 mM dNTP, 0.5 μM each forward and reverse primers, and 2.5 Units Taq polymerase. The sequences of the primers used are as follows:

1) For double-stranded ends:

a) Lis221nomF -
(SEQ ID No. 6)
5′-AAAAAAATCACTCTGGAGGATACGTTGC-3′
and
b) Lis221nomR -
(SEQ ID No. 7)
5′-AAAAAAATTACCGTTCTCCACCATTCC-3′;

2) For no ‘DNA label’:

a) Lis221nhF -
(SEQ ID No. 8)
5′-TCACTCTGGAGGATACGTTGC-3′
and
b) Lis221nhR -
(SEQ ID No. 9)
5′-TTACCGTTCTCCACCATTCC-3′;

3) For single-stranded ends:

a) Lis221F -
(SEQ ID No. 10)
5′-AAAAAAmATCACTCTGGAGGATACGTTGC-3′
and
b) Lis221R -
(SEQ ID No. 11)
5′-AAAAAAmATTACCGTTCTCCACCATTCC-3′.

The sequence of the complementary capture probe on the latex bead is 5′-TTTTTTTTTTTTTTTTTTTT-biotin3′ (SEQ ID No. 12). The target nucleic acid is a plasmid containing the cDNA of the Listeria monocytogenes hemolysin gene. (Genbank accession no. FJ263386).

A PCR reaction was then performed by incubating at 94° C. for 2 minutes followed by 30 cycles of 94° C. for 30 seconds, 55° C. for 30 seconds, 72° C. for 1 minutes, and a final extension at 72° C. for two minutes. Other incubation times and/or cycles can be used in other embodiments, depending on the primers used and the desired amount of amplification. An aliquot of the reaction was mixed with the latex-complementary nucleic acid sequence reagent for 2-30 minutes at room temperature. For the latex agglutination assays, 30 uL of the PCR sample and 5 uL of the 6.25 mg/mL oligo-latex bead was applied to the latex card, mixed with a pipet tip, and incubated for 10 minutes. The resulting aggregation, which is indicative of the presence of PCR product, is inspected visually, on a card (see FIG. 6).

### Example 6

### Detection of RCA Product

Some embodiments of the methods disclosed herein use latex beads coupled to single stranded DNA which is complementary to a region designed as part of an RCA product. The complementary region, which is coupled to the latex bead, captures the RCA product. The RCA product is produced by elongation of a primer (target nucleic acid) hybridized to a circular probe. The circular probe is created by hybridization of the target nucleic acid and addition of T4 DNA ligase. In some embodiments, the target nucleic acid is incubated in 50 mM Tris-HCl (pH 7.5), 10 mM MgCl2, 10 mM DTT, 1 mM ATP, 100 fmol let 7d phosphorylated linear DNA padlock probe for 3 minutes at 65C. The sequence of the nucleic acids used in these reactions are as follows:

a) let7d padlock probe:
(SEQ ID No. 13)
5′P-CTACTACCTC TTTTATTTCC TCAATGCTGC TGCTGTACTA CTAGTGATTT
ACTTGGATGT CTAACTATGC AAA-3′,
b) let 7d mutant padlock probe -
(SEQ ID No. 14)
5′P-CTAGTACCTCTT TTATTTCCTC AATGCTGCTG TACTACTAGT GATTTACTTG
GATGTCTAAC TATGGAAC-3′,
c) let 7d target ribonucleic acid -
(SEQ ID No. 15)
5′AGAGGUAGUAGGUUGCAUAGUU-3′.
SEQ ID Nos. 13-16 were derived from human sequences.

The sequence of the complementary capture probe on the latex bead is 5′-CTACTACCTCTAAAAA-biotin-3′ (SEQ ID No. 16). The sample is allowed to cool to room temperature and 400 U T4 DNA ligase or none (let 7d-ligase) is added and allowed to incubate at 37° C. for 2 hours. After heat inactivation at 65° C. for 10 minutes, a 10 uL aliquot of the sample was added to 50 mM Tris-HCl (pH 7.5), 10 mM MgCl2, 10 mM (NH4)2SO4, 4 mM DTT, 10 U phi 29 DNA polymerase, 200 nM dNTP and 200 ug/mL BSA. The reactions were then incubated overnight at 30° C. followed by 10 minutes at 65° C. to inactivate the polymerase. For the latex agglutination assays, 25 uL of the RCA sample and 5 uL of the 6.25 mg/mL oligo-latex bead was applied to the latex card and mixed using a pipet tip. Agglutination was observed by 10 minutes (see FIG. 7).

1. A method for detecting the presence of an amplified nucleic acid comprising:
amplifying a sample of a specific target nucleic acid under amplification conditions that incorporate a detectable label into said sample of said target nucleic acid during said amplification;
contacting said specific target nucleic acid samples with a sample of a capture matrix,
wherein said capture matrix comprises a solid support coupled to an agent that recognizes said detectable label,
wherein recognition of said detectable label by said agent induces agglutination of said specific target nucleic acid with said capture matrix and causes an associated increase in turbidity of the specific target nucleic acid sample; and
detecting the presence of said amplified target nucleic acid by detecting said increased turbidity in said specific target sample.

2. The method of claim 1, further comprising subjecting a sample of control nucleic acid that does not include said specific target nucleic acid to said amplification conditions;
contacting said control nucleic acid sample with a sample of a capture matrix,
wherein contacting of said control nucleic acid sample with said capture matrix produces a control level of induction of agglutination and associated alteration of turbidity of the sample; and
detecting the presence of said amplified target nucleic acid by detecting said increased turbidity in said specific target sample as compared to said control sample

3. The method of claim 1, wherein said contacting of said specific target nucleic acid sample with said sample of a capture matrix occurs prior to said amplification.

4. The method of claim 1, further comprising:
quantifying the amount of amplified specific target nucleic acid by comparing the turbidity of said specific target sample with a standard curve,
wherein said standard curve is generated by amplifying a plurality of known amounts of a nucleic acid comprising a detectable label to generate a plurality of samples and contacting each of said plurality of samples with a sample of capture matrix, thereby generating a standard curve of turbidity corresponding to said known amounts of nucleic acid.

5. The method of claim 1 wherein the solid support is comprises latex beads.

6. The method of claim 1, wherein said solid support is coupled to an antibody that interacts with said detectable label.

7. The method of claim 6, wherein said detectable label is 5-methyl-cytosine and wherein said antibody is reactive to 5-methyl-cytosine.

8. The method of claim 1, wherein said amplification of said specific target nucleic acid comprises using at least one biotin-labeled primer.

9. The method of claim 8, further comprising concentrating said amplified specific target nucleic acid by contacting said amplified specific target nucleic acid with streptavidin coupled to a solid support and washing said concentrated amplified specific target nucleic acid to remove excess detectable label prior to said contacting with said capture matrix.

10. (canceled)

11. The method of claim 1, wherein said detectable label are incorporating by amplifying said specific target nucleic acid using one or more modified primers with a tag sequence.

12. The method of claim 11, wherein said modified primers comprises a gene-specific region, a chain terminating nucleotide, and a nucleic acid label comprising 6-20 nucleotides.

13. The method of claim 12, wherein said nucleic acid label is recognized by said agent and wherein said agent comprises a plurality of oligonucleotides at least partially complementary to said nucleic acid label.

14. (canceled)

15. The method of claim 12, wherein said nucleic acid label is selected from the group consisting of oligonucleotide labels, single-stranded DNA labels, RNA labels, peptide nucleic acid labels, locked nucleic acid labels, glycol nucleic acid labels, threose nucleic acid labels, or combinations thereof

16. The method of claim 1 wherein amplification of nucleic acid is performed using nucleotides at a concentration between 1 and 50 μM.

17. (canceled)

18. The method of claim 1 wherein the nucleic acid is selected from the group consisting of DNA, RNA, siRNA, tRNA, and snRNA.

19. The method of claim 1 wherein the method of amplifying nucleic acid is selected from the group consisting of the polymerase chain reaction, rolling circle amplification, nucleic acid sequence based amplification, transcription mediated amplification, and ligase chain reaction.

20. The method of claim 1, wherein said detectable label comprises at least one target specific primer comprising at least one 2′ O-methyl RNA base, and
wherein said agent comprises a single stranded nucleic acid complementary to a region on said target specific primer.

21. The method of claim 20, wherein said single stranded nucleic acid is selected from a group consisting of oligonucleotides, single-stranded DNA, RNA, peptide nucleic acid, locked nucleic acid, glycol nucleic acid, and threose nucleic acid.

22. The method of claim 20, wherein the solid support is latex beads.

23. The method of claim 20, wherein at least one target specific primer is coupled to a solid support.


SEQUENCE LISTING (16 sequences)

SEQ ID NO: 1 - DNA, 20 bp, Listeria monocytogenes
  misc_feature: Forward Primer
cgtccatctatttgccaggt

SEQ ID NO: 2 - DNA, 20 bp, Listeria monocytogenes
  misc_feature: Reverse Primer
atttcggataaagcgtggtg

SEQ ID NO: 3 - DNA, 28 bp, Listeria monocytogenes
  misc_feature: Forward 2′O-methylated RNA primer
aaaaaaatcactctggaggatacgttgc

SEQ ID NO: 4 - DNA, 27 bp, Listeria monocytogenes
  misc_feature: Reverse 2′O-methylated RNA primer
aaaaaaattaccgttctccaccattcc

SEQ ID NO: 5 - DNA, 20 bp, Listeria monocytogenes
  misc_feature: Capture probe
tttttttttttttttttttt

SEQ ID NO: 6 - DNA, 28 bp, Listeria monocytogenes
  misc_feature: Forward “No methyl” primer
aaaaaaatcactctggaggatacgttgc

SEQ ID NO: 7 - DNA, 27 bp, Listeria monocytogenes
  misc_feature: Reverse “No Methyl” primer
aaaaaaattaccgttctccaccattcc

SEQ ID NO: 8 - DNA, 21 bp, Listeria monocytogenes
  misc_feature: Forward “no handle” primer
tcactctggaggatacgttgc

SEQ ID NO: 9 - DNA, 20 bp, Listeria monocytogenes
  misc_feature: Reverse “no handle” primer
ttaccgttctccaccattcc

SEQ ID NO: 10 - DNA, 28 bp, Listeria monocytogenes
  misc_feature: Forward “Single Stranded Ends” primer
aaaaaaatcactctggaggatacgttgc

SEQ ID NO: 11 - DNA, 28 bp, Listeria monocytogenes
  misc_feature: Reverse “Single Stranded Ends” primer
aaaaaamattaccgttctccaccattcc

SEQ ID NO: 12 - DNA, 20 bp, Listeria monocytogenes
  misc_feature: Capture probe
tttttttttttttttttttt

SEQ ID NO: 13 - DNA, 73 bp, Homo sapiens
  misc_feature: Let-7d padlock probe
ctactacctcttttatttcctcaatgctgctgctgtactactagtgatttacttggatgtctaactatgcaaa

SEQ ID NO: 14 - DNA, 70 bp, Homo sapiens
  misc_feature: Let-7d mutant padlock probe
ctagtacctcttttatttcctcaatgctgctgtactactagtgatttacttggatgtctaactatggaac

SEQ ID NO: 15 - RNA, 22 nt, Homo sapiens
  misc_feature: Let-7d target ribonucleic acid
agagguaguagguugcauaguu

SEQ ID NO: 16 - DNA, 16 bp, Homo sapiens
  misc_feature: Let-7d capture probe
ctactacctctaaaaa